(19) United States
(12) Patent Application Publication
(10) Pub. No.: US 2006/0189890 A1
(43) Pub. Date: Aug. 24, 2006

(54) Liquid handling means for excision apparatus

(76) Inventors: Andrew Arthur Gooley, New South Wales (AU); Robert Ramsden, New South Wales (AU); Kurt Weber, New South Wales (AU)

Correspondence Address: KATTEN MUCHIN ROSENMAN LLP, 575 MADISON AVENUE, NEW YORK, NY, 10022-2585, US

(21) Appl. No.: 10/723,990

(22) Filed: Nov. 25, 2003

Related U.S. Application Data
(63) Continuation of application No. PCT/AU02/00657, filed on May 27, 2002.

(30) Foreign Application Priority Data
May 25, 2001 (AU) PR5273

(51) Int. Cl. A61B 10/00

(52) U.S. Cl. 600/562

(57) ABSTRACT

A device for dispensing liquids includes one or more liquid outlet means carried on a movable head. The device forms part of an automated excision apparatus for cutting biomolecule spots from an array of such spots carried in a gel. The head is mounted on an automated motion control system to allow for movement of the head in X, Y, Z directions under the control of a control means. A displacement means for causing the dispensing of liquid from the one or more liquid outlet means of the device is also carried by the moveable head carrying the liquid delivery outlets allowing the length of the tubing between the outlet and the aspiration device, typically a syringe, to be much shorter. This means that swelling and contraction of the tube is minimized during liquid handling operations which means more accurate delivery of specified volumes through the outlet. The syringes and the outlets are mounted on opposite sides of an axis of movement of the apparatus, keeping the machine compact. Typically the axis will be a horizontal X or vertical Y axis defined by a beam or the like which supports the movable head.

### CROSS-REFERENCE TO RELATED APPLICATION

The present application is a continuation of International Application No. PCT/AU02/00657, which was filed on May 27, 2002.

### FIELD OF THE INVENTION

This invention relates to a liquid delivery and aspiration (i.e. fluid pick-up and delivery) device and, in particular, to a liquid delivery and aspiration device associated with an automated control system which allows minimum tubing connection between the outlet of the liquid delivery and aspiration device and a source of positive displacement, typically a syringe.

### BACKGROUND OF THE INVENTION

Liquid handling devices are used in laboratories in the processing of biomolecules such as proteins, peptides, saccharides, lipids, nucleic acid molecules and complex biomolecules such as glycoproteins. They are used to remotely wash, add reagents to, suck up liquid from, a sample biomolecule which may be a spot cut from an array of spots from a 2d gel. The reagents are generally held in microtitre plates (MTP) and are sucked up into a pipette known as a “tip” (or a “zip tip” if it contains a chromatographic resin) and then robotically moved to a further MTP which contains the sample or samples. The particular sequence of reagents and treatments applied depends on the biomolecule and the desired end result. The biomolecule may for example be a protein being prepared (digested) for MALDI-TOF analysis. Typically, automated liquid handling devices have their syringes mounted on a fixed platform remote from the operating area of the instrument. This necessitates the use of extensive lengths of tubing between the syringes and the point of liquid delivery, i.e. the tips.

The problem with this approach, is that the tubes must be carried with the moving parts of the liquid handling devices which is generally awkward. Further the tubing becomes contaminated with liquids and is difficult to clean and replace.

A second problem is that the length of tubing makes it difficult to accurately control delivery of liquid from the outlet of the devices because of the inertia of the system due to expansion of the tubing, air bubbles in the tubing, and the like.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

The present invention seeks to provide an improved liquid delivery and aspiration device which addresses the problems of the prior art discussed above.

### SUMMARY OF THE INVENTION

Thus according to the present invention, there is provided a sample cutting and handling apparatus for excising one or more samples from an array of samples including:

at least one cutting head mounted on the apparatus for movement in X, Y and Z directions by an automated control system for excising a sample from the array;

one or more liquid aspiration and delivery outlet means for dispensing liquids onto the array of samples;

a movable head mounted on an automated motion control system for controlling movement of the moveable head in X, Y, and Z directions;

at least one displacement means connected to a liquid supply means and to one or more of the liquid aspiration and delivery outlet means via a valve means for causing the dispensing of liquid from the one or more liquid aspiration and delivery outlet means;

wherein at least one displacement means and the one or more liquid aspiration and delivery outlet means are carried by the moveable head.

The fact that the displacement means is carried on the head, allows the length of tubing between the outlet and the aspiration device, typically a syringe, to be much shorter, than has previously been possible thus making tubing maintenance and replacement much simpler. Furthermore, the shorter length of tubing means that swelling and contraction of the tube is minimized during liquid handling operations which in turn results in much more accurate delivery of specified volumes through the outlet, particularly with sub-5 microlitre volumes. Ordinarily to achieve such accurate delivery of specified volumes using the prior art methods of remote aspiration devices, it is necessary to use syringes having a volume of approximately 100 microlitres in order to give the level of precision required. However, 100 microlitre syringes are not robust and require frequent maintenance and replacement. With the system of the present invention, larger 1 mL syringes can be successfully used. Such 1 mL syringes are more robust than 100 microlitre syringes and last much longer before requiring replacement.

A reservoir connected to the movable head by a single tube may supply liquid (typically water) to the syringes.

In a particularly preferred embodiment, the syringes and the outlets are mounted on opposite sides of an axis of movement of the apparatus. Typically the axis will be a horizontal X or Y axis defined by a beam or the like which supports the movable head. Mounting the syringes on one side of the axis and the outlets on the other, enables the machine to be compact.

Typically a bank of eight syringes and a bank of eight liquid outlet means are mounted either side of the movable head. In use pipettes or tips are attached to the liquid outlet means which may simply be an open end of tubing. These tips are used to pick up and dispense reagents, wash and the like. The tips are discarded and replaced with clean tips, as required. “Zip tips” which include resin may also be used. In a preferred embodiment, the eight syringes are mounted on parallel axes which, in use, are generally vertically oriented. However the system is sufficiently robust to allow for much larger arrays of syringes, for example eight columns of twelve syringes.

The device for dispensing liquids preferably forms an element of a sample cutting and handling apparatus disclosed in an international patent application filed in the name of Shimadzu Corporation and Proteome Systems Pty Ltd on 27 May 2002 entitled “Sample Collection and Preparation Apparatus” the contents of which are incorporated herein by reference. The device for dispensing liquids is located on the same machine head as a cutting head and moves up and down in the z-axis with the same. In a preferred embodiment, a clutch means is provided on the axis of movement of the syringes which when disengaged fixes the cylinders relative to the pistons of the syringes so that as the head moves up and down the piston and syringe body move together, and when engaged releases the body of the syringe relative to the piston such that as the module moves up and down in the vertical direction, the piston moves relative to the syringe body. Valves are provided for controlling the flow of liquid through the outlets however, the clutch reduces wear in the liquid handling syringes hence the clutch disengages during gel cutting.

### BRIEF DESCRIPTION OF THE DRAWINGS

A specific embodiment of the invention will now be described, by way of example only, and with reference to the accompanying drawings in which:

FIG. 1 is a side elevation of a sample cutting and preparation apparatus including a liquid handling device for dispensing and aspirating liquids embodying the present invention;

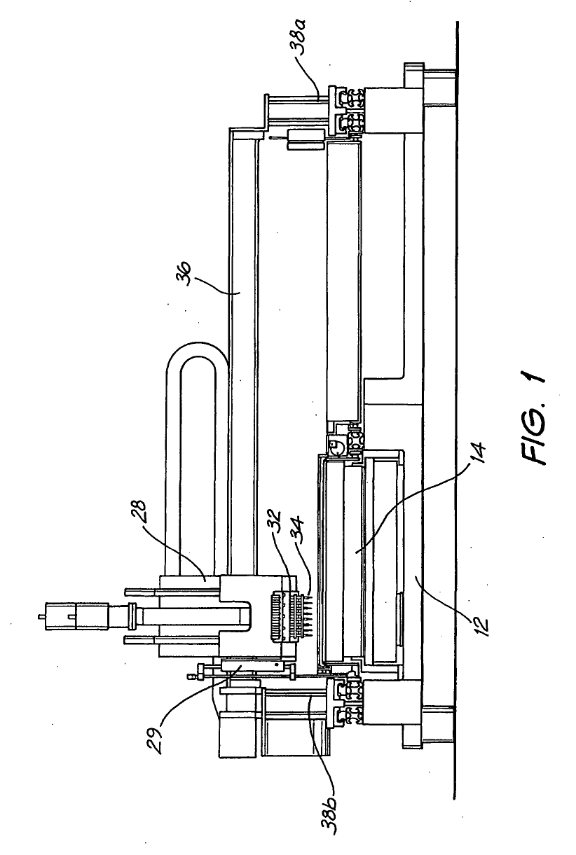

FIG. 2 is a plan view of the apparatus shown in FIG. 1;

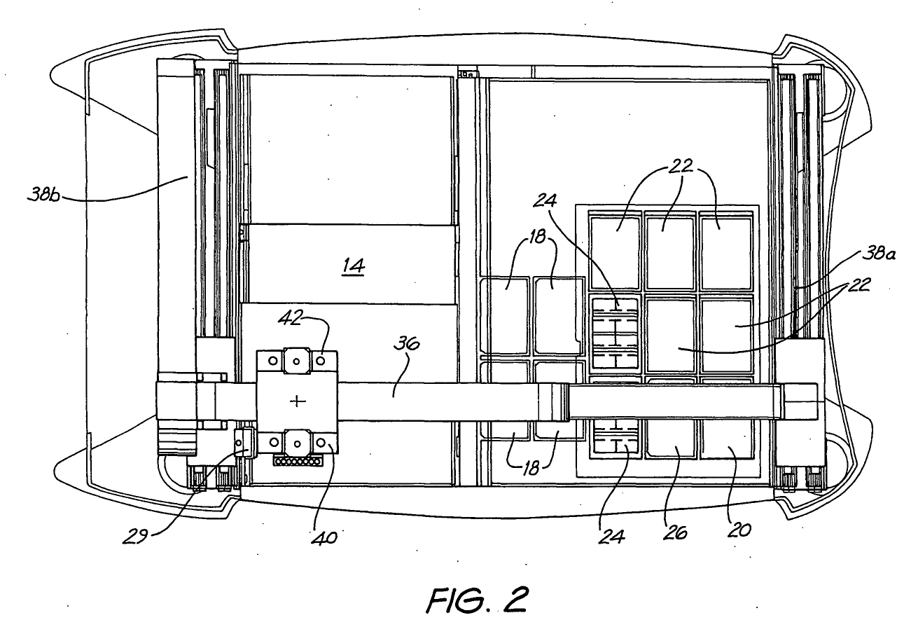

FIG. 3 is a schematic view of a syringe and bi-directional valve;

FIG. 4 is a front view of the liquid handling device; and

FIG. 5 is a rear view of the liquid handling device of FIG. 4.

### DETAILED DESCRIPTION OF A PREFERRED EMBODIMENT

Referring to the drawings, the apparatus generally indicated at 10 includes a chassis 12 on which a scanner 14 is supported. The scanner may be a high resolution “desktop” type scanner and includes a glass table 16 above which a two dimensional array of biomolecules such as proteins in a gel or supported on a membrane can be placed. Adjacent the scanner, there are four microtitre plates (MTPs) 18, a 384 well MALDI target plate 20, boxes 22 containing pipette tips and/or “zip tips” 30, trays 24 containing various solvents and reagents and a 384 well MTP 26 containing purified water.

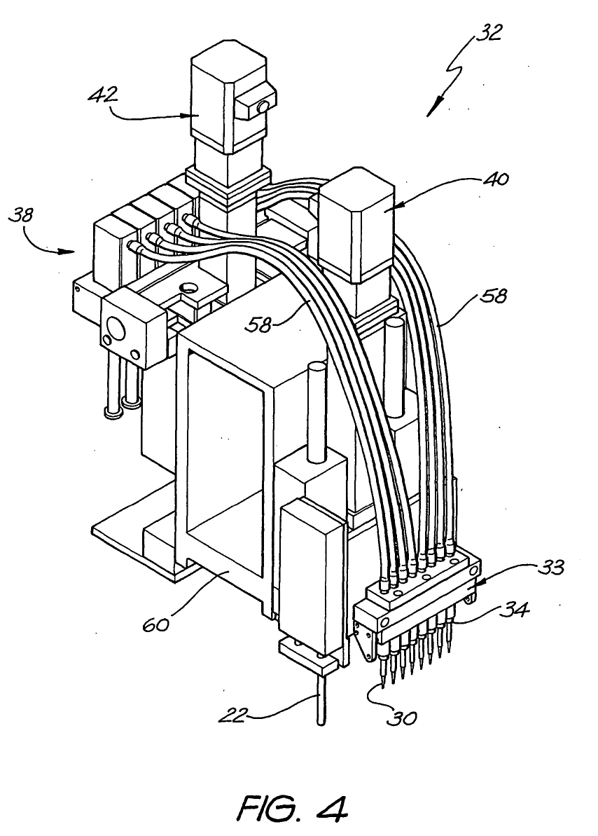

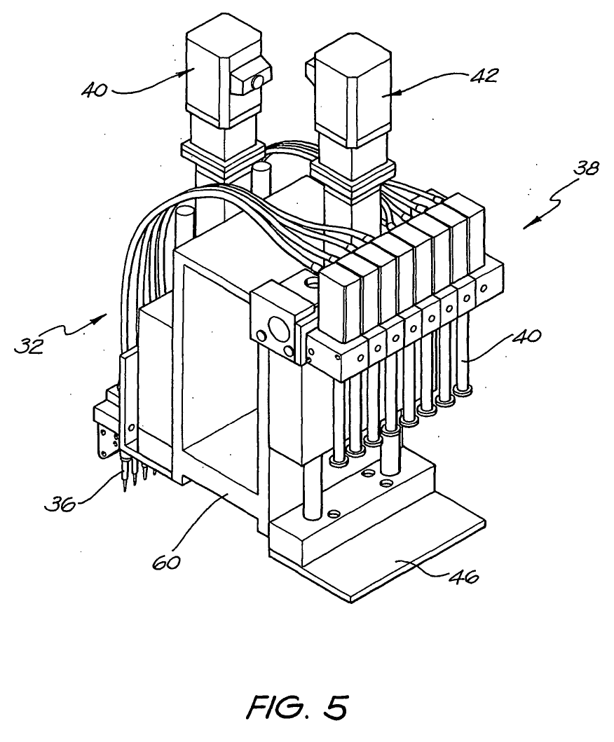

Mounted for movement above the scanner, is a machine head 28 including a cutting tool 29 and an eight channel liquid delivery means 32 (best seen in FIGS. 4 and 5) including eight adjacent liquid delivery outlets 34. The machine head moves along an X axis 36 which in turn extends between and is supported by two Y axes 38a, 38b. The machine head includes a Z axis driver 40 for driving the cutting tool up and down in a vertical direction and a parallel W axis 42 for driving liquid delivery means. Thus, the machine head can move not only in mutually perpendicular horizontal X and Y directions parallel to the surface of the base frame, but can also move in the vertical Z direction so that a specified portion of an array supported on the glass table 16 can be cut and picked up by the cutting tool 29. The machine head may also be moved so that liquid delivery tips 30 carried by the liquid delivery outlets 34 are positioned to aspirate reagents/solvents from the solvent trays 24 or MTP 26 and to dispense the aspirated reagents/solvents onto samples which have been excised from the gel and placed in microtitre plates 18. It is also possible to dispense reagents onto the samples in situ in the gel. The vertical height of the tips 30 is also controlled by the Z axis. A control means, not illustrated, controls the operation of the apparatus.

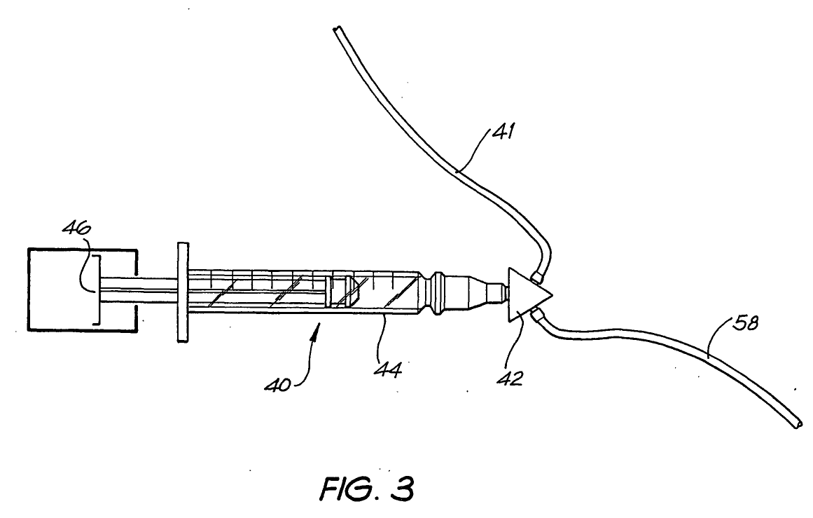

The liquid delivery means 32 includes a pipette carrier 33 which carries the eight liquid delivery outlets 34. The outlets can receive either plain tips or zip tips 30. Each liquid delivery outlet is connected to a liquid handler 38 by a short length of tubing 58 so as to be able to dispense and aspirate water. The liquid handler comprises eight parallel bi-directional liquid-handling syringes 40. As is schematically illustrated in FIG. 3 each syringe is connected via a bi-directional valve 42 to tubing 41 connected to a reservoir or to tubing 58 connected to one of the probes as shown in FIG. 3, depending on the state of the valve. The body of the syringe is fixed and the actuator/piston rod 48 is moveably by a W axis actuator 48 which draws liquid into, and expels liquid from the syringe.

The liquid-handling syringes are ganged together and actuated by the W axis robot. A gel cutting syringe which supplies water to the gel during cutting operations is also ganged together with the liquid-handling syringes. The valves operate in parallel under control of the control means. The syringes are connected to the W axis via a clutch. All the syringes are actuated by the same robot. If the clutch were not provided the liquid-handling syringes would operate every time the gel cutting syringe operated. Although this would not affect the process of liquid handling because the valves determine the fluid flow, it would result in substantially more wear and tear on the liquid-handling syringes hence the clutch disengages during gel cuffing. In an alternative embodiment however the clutch can be omitted.

Two reservoirs which supply liquid (typically purified water) to the syringes are situated at the highest point in the instrument maximizing the effect of gravity feed.

Feeds are done via tubes which follows the control cable guides used for controlling the moving W and Z axis. One reservoir feed is a single tube going into a manifold connecting the 8 syringe pumps on the W axis 42. The second reservoir feed is also by a single tube connecting to the cutting head syringe pump located on the W axis actuator 42.

As discussed above, from the W axis a set of tubes 58 connect the syringes to the cutting head and pipette tips mounted on the Z axis. The tubes pass over the mounting block 60. This is to achieve the shortest path possible between pumps and tips. The co-location of the syringe pumps and the tips on the same moving head minimizes bubbles and hydraulic effects which occurs with long tube paths between pumps and tips.

During the sample separation process, the probes will “dip and sip” appropriate solutions from a well located on the right drawer.

To dispense wastes the probes moves to a waste point also on the right drawer where waste solutions will exits the drawer and be siphoned out of the instrument.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A sample cutting and handling apparatus for excising one or more samples from an array of samples including:
at least one cutting head mounted on the apparatus for movement in X, Y and Z directions by an automated control system for excising a sample from the array;
one or more liquid aspiration and delivery outlet means for dispensing liquids onto the array of samples;
a movable head mounted on an automated motion control system for controlling movement of the moveable head in X, Y, and Z directions;
at least one displacement means connected to a liquid supply means and to one or more of the liquid aspiration and delivery outlet means via a valve means for causing the dispensing of liquid from the one or more liquid aspiration and delivery outlet means;
wherein at least one displacement means and the one or more liquid aspiration and delivery outlet means are carried by the moveable head.

2. The sample cutting and handling apparatus for dispensing liquids as claimed in claim 1 wherein at least one displacement means comprises a syringe and at least one liquid aspiration and delivery outlet means comprises a pipette which is capable of receiving a removable tip.

3. The sample cutting and handling apparatus for dispensing liquids as claimed in claim 2 wherein a plurality of syringes are mounted in parallel on the movable head.

4. The sample cutting and handling apparatus for dispensing liquids as claimed in claim 3 wherein the syringes and the outlets are mounted on opposite sides of an axis of movement of the apparatus along which the movable head is arranged to travel.

5. The sample cutting and handling apparatus for dispensing liquids as claimed in claim 1 wherein a reservoir is connected to the movable head by a single tube for supplying liquid to at least one displacement means.

6. The sample cutting and handling apparatus for dispensing liquids as claimed in claim 4 wherein eight or more syringes are mounted along parallel axes which, in use, are generally vertically oriented.

7. The sample cutting and handling apparatus for dispensing liquids as claimed in claim 1 wherein the device for dispensing liquids is located on the same movable head as at least one cutting head.

8. The sample cutting and handling apparatus for dispensing liquids as claimed in claim 2 wherein a clutch means is provided on the axis of movement of the syringes which when disengaged fixes the cylinders relative to the pistons of the syringes so that as the head moves up and down the piston and syringe body move together, and when engaged releases the body of the syringe relative to the piston such that as the module moves up and down in the vertical direction, the piston moves relative to the syringe body.

9. The sample cutting and handling apparatus for dispensing liquids as claimed in claim 2 wherein one or more valves are provided for controlling the flow of liquid to and from the syringes.

10. A sample cutting and handling apparatus for excising one or more samples from an array of samples including:
at least one cutting head mounted on the apparatus for movement in X, Y and Z directions by an automated control system for excising a sample from the array;
a plurality of pipettes for dispensing liquids onto the array of samples;
a movable head mounted on an automated motion control system for controlling movement of the moveable head in X, Y and Z directions;
a plurality of syringes, each syringe being connected to a liquid supply means and to one of the pipettes via a valve means for causing the dispensing of liquid from the one or more liquid aspiration and delivery outlet means;
wherein both the syringes and the pipettes are carried by the moveable head, and wherein the syringes and the pipettes are mounted on opposite sides of the x-axis of movement of the apparatus along which x-axis the movable head is arranged to travel.

11. The sample cutting and handling apparatus for dispensing liquids as claimed in claim 10 wherein a reservoir is connected to the movable head by a single tube for supplying liquid to the syringes.

12. The sample cutting and handling apparatus for dispensing liquids as claimed in claim 11 wherein the device for dispensing liquids is located on the same moveable head as at least one cutting head.

13. A sample cutting and handling apparatus for excising one or more samples from an array of samples including:
at least one cutting head mounted on the apparatus for movement in X, Y and Z directions by an automated control system for excising a sample from the array;
one or more liquid aspiration and delivery outlet means for dispensing liquids onto the array of samples;
a movable head mounted on an automated motion control system for controlling movement of the moveable head in X, Y and Z directions;
a plurality of syringes connected to a liquid supply means, each syringe also being connected to one of the liquid aspiration and delivery outlet means via a valve means for causing the dispensing of liquid from the one or more liquid aspiration and delivery outlet means;
wherein both the syringes and the one or more liquid aspiration and delivery outlet means are carried by the moveable head, wherein the syringes and the outlets are mounted on opposite sides of an x axis of movement of the apparatus along which the moveable head is arranged to travel; and
wherein a reservoir is connected to the moveable head by a single tube for supplying liquid to at least one displacement means.

14. The sample cutting and handling apparatus for dispensing liquids as claimed in claim 13 wherein the device for dispensing liquids is located on the same movable head as at least one cutting head.
